(19) United States
(12) Patent Application Publication
(10) Pub. No.: US 2022/0309296 A1
(43) Pub. Date: Sep. 29, 2022

(54) A SYSTEM AND METHOD FOR GENERATING AND INTERACTING WITH INTERACTIVE MULTILAYERED DATA MODELS

(71) Applicant: POMICELL LTD., Jerusalem (IL)

(72) Inventors: Boaz BUCHANDLER, Jerusalem (IL); Moshe SHACHOR, Jerusalem (IL); Amram BEN DAVID, Jerusalem (IL)

(73) Assignee: POMICELL LTD., Jerusalem (IL)

(21) Appl. No.: 17/261,583

(22) PCT Filed: Nov. 4, 2020
(86) PCT No.: PCT/IL2020/051150

§ 371 Date: Jan. 20, 2021

Related U.S. Application Data
(60) Provisional application No. 62/930,614, filed on Nov. 5, 2019.

(51) Int. Cl. G06K 9/62; G06N 5/02; G06N 20/00; G06F 30/27

(52) U.S. Cl. CPC G06K 9/6289; G06N 5/022; G06N 5/025; G06N 20/00; G06F 30/27

(57) ABSTRACT

Disclosed herein is a method comprising using at least one hardware processor for obtaining raw data relating to at least one predetermined field of study, obtaining at least one term, generating at least a first entity and a second entity from said raw data according to said at least one term, implementing a predetermined function to determine a relationship between said first and second entities, generating a layer aligning said first and second entities according to the relationship between said first and second entities, and constructing a model of data-interactions according to said at least one interactive map layer.

### RELATED APPLICATIONS

The present application claims priority from U.S. Provisional Application Ser. No. 62/930,614 filed on Nov. 5, 2019.

### FIELD OF THE INVENTION

The present disclosure generally relates to modeling data.

### BACKGROUND

There is a wide and varied assortment of computational models that aim to describe and predict via simulation environment elements, interactions and cross-effects of different structures in various fields of study. Various fields of study include information that is used to improve what is known in a particular field of study and to achieve new findings and innovations. Graphs, maps, charts and models can be used to represent parts of this information in a clear and “user-friendly” manner. Professionals in the field of study rely on the old information to make new findings that improve, progress and advance the knowledge in the field of study.

### SUMMARY

The following embodiments and aspects thereof are described and illustrated in conjunction with systems, tools and methods which are meant to be exemplary and illustrative, not limiting in scope.

There is provided, in accordance with an embodiment, a method comprising using at least one hardware processor for obtaining raw data relating to at least one predetermined field of study; obtaining at least one term generating at least a first entity and a second entity from the raw data according to the at least one term, implementing a predetermined function to determine a relationship between the first and second entities, generating a layer aligning the first and second entities according to the relationship between the first and second entities, and constructing a model of data-interactions according to the at least one interactive map layer.

In some embodiments, implementing the predetermined function comprises implementing a training function configured to determine a relationship between at least two entities via computational simulations or other mathematical translational method in view of experimental data.

In some embodiments, each entity comprises an abstract research significance related to a collection of the raw data.

In some embodiments, the predetermined function is an insight function for determining at least one insight having an optimal translation in terms of required usage of at least one entity of the at least first and second entities.

In some embodiments, the at least one map layer comprises a collection of the plurality of entities having a predetermined relationship of interaction networks between them.

In some embodiments, the model comprises a complete research collection having a well-defined collection of entities, layers and functions customized to a requirement of a user.

In some embodiments, the predetermined function is a distance-function for determining mathematical representations of cause- and effect measurements, in a unifying manner between the first and second entity.

In some embodiments, the model relates to a predetermined field of technology.

In some embodiments, the method further includes parsing the raw data according to the at least one term

In some embodiments, the at least one term is a seeded term.

In some embodiments, the at least one term is an input provided by a user.

In some embodiments, the at least one map layer comprises an interface providing at least recommendation of actual research and analysis real-world action and experiment.

In some embodiments, the at least one map layer comprises a business intelligence interface for providing a background about at least one field of study.

In some embodiments, the method further includes obtaining data relating to a product, determining a mechanism of the product, and utilizing a research mechanism to produce a recommended usage of the product according to the at least one insight.

In some embodiments, the method further includes executing a train function to automatically and continuously update the model with according to new data and inputs.

In some embodiments, the model is displayed with entities represented by nodes interconnected by pathways representing the relationship between the entities.

In some embodiments, the method further includes executing a data crawling of at least one database to obtain the raw data, wherein the at least one database is selected from a database library.

In some embodiments, each entity is allocated a unique location within the model according to execution of a distance function, wherein the location of each entity is updated to provide a finite location within the model.

In some embodiments, the at least one entity can provide a representation of a model derivation having parts of a source model and providing additional updates to distances according to the training of the model.

There is further provided, in accordance with an embodiment, a computer program product for generating an interactive data-transfer and analysis computational model, the computer program product comprising a non-transitory computer-readable storage medium having program code embodied therewith, the program code executable by at least one hardware processor to obtain raw data relating to at least one predetermined field of study, obtain at least one term, generate at least a first entity and a second entity from the raw data according to the at least one term, implement a predetermined function to determine a relationship between the first and second entities, generate a layer aligning the first and second entities according to the relationship between the first and second entities, and construct a model of data-interactions according to the at least one interactive map layer.

In some embodiments, implementing the predetermined function comprises implementing a training function configured to determine a relationship between at least two entities via computational simulations or other mathematical translational method in view of experimental data.

In some embodiments, each entity comprises an abstract research significance related to a collection of the raw data.

In some embodiments, the predetermined function is an insight function for determining at least one insight having an optimal translation in terms of required usage of at least one entity of the at least first and second entities.

In some embodiments, the at least one map layer comprises a collection of the plurality of entities having a predetermined relationship of interaction networks between them.

In some embodiments, the model comprises a complete research collection having a well-defined collection of entities, layers and functions customized to a requirement of a user.

In some embodiments, the predetermined function is a distance-function for determining mathematical representations of cause- and effect measurements, in a unifying manner between the first and second entity.

In some embodiments, the model relates to a predetermined field of technology.

In some embodiments, the computer program product further includes parsing the raw data according to the at least one term.

In some embodiments, at least one term is a seeded term.

In some embodiments, the at least one term is an input provided by a user.

In some embodiments, the at least one map layer comprises a research-navigation interface providing at least recommendation of actual research and analysis real-world action and experiment.

In some embodiments, the at least one map layer comprises a business intelligence interface for providing a background about at least one field of study.

In some embodiments, the computer program product further includes obtaining data relating to a product, determining a mechanism of the product, and utilizing a research mechanism to produce a use recommendation of the product according to the at least one insight.

In some embodiments, the computer program product further includes executing a train function to automatically and continuously update the model with according to new data and inputs.

In some embodiments, the model is displayed with entities represented by nodes interconnected by pathways representing the relationship between the entities.

In some embodiments, the computer program product further comprising executing a data crawling of at least one database to obtain the raw data, wherein the at least one database is selected from a database library.

In some embodiments, each entity is allocated a unique location within the model according to execution of a distance function, wherein the location of each entity is updated to provide a finite location within the model.

In some embodiments, the at least one entity can provide a representation of a model derivation having parts of a source model and providing additional updates to distances according to the training of the model.

There is further provided, in accordance with an embodiment, a system including at least one database for storing raw data, a mapping server configured to obtain raw data relating to at least one predetermined field of study, obtain at least one term, generate at least a first entity and a second entity from the raw data according to the at least one term, implement a predetermined function to determine a relationship between the first and second entities, generate a layer aligning the first and second entities according to the relationship between the first and second entities, and construct a model of data-interactions according to the at least one interactive map layer, and a computer having a user interface for displaying the model and to enable a user to interact with the model.

In some embodiments, implementing the predetermined function comprises implementing a training function configured to determine a relationship between at least two entities via computational simulations or other mathematical translational method in view of experimental data.

In some embodiments, each entity comprises an abstract research significance related to a collection of the raw data.

In some embodiments, the predetermined function is an insight function for determining an optimal translation in terms of required usage of at least one entity of the at least first and second entities.

In some embodiments, the at least one map layer comprises a collection of the plurality of entities having a predetermined relationship of interaction networks between them.

In some embodiments, the model comprises a complete research collection having a well defined collection of entities, layers and functions customized to a requirement of a user.

In some embodiments, the predetermined function is a distance-function for determining mathematical representations of cause- and effect measurements, in a unifying manner between the first and second entity.

In some embodiments, the model relates to a predetermined field of technology.

In some embodiments, the mapping server is further configured to parse the raw data according to the at least one term.

In some embodiments, the at least one term is a seeded term.

In some embodiments, the at least one term is an input provided by a user.

In some embodiments, the at least one map layer comprises a research-navigation interface providing at least recommendation of actual research and analysis real-world action and experiment.

In some embodiments, the at least one map layer comprises a business intelligence interface for providing a background about at least one field of study.

In some embodiments, the mapping server is further configured to obtain data relating to a product, determine a mechanism of the product, utilize a research mechanism to produce a use recommendation of the product according to the at least one insight.

In some embodiments, the mapping server is further configured to execute a train function to automatically and continuously update the model with according to new data and inputs.

In some embodiments, the model is displayed with entities represented by nodes interconnected by pathways representing the relationship between the entities.

In some embodiments, the mapping server is further configured execute a data crawling of at least one database to obtain the raw data, wherein the at least one database is selected from a database library.

In some embodiments, each entity is allocated a unique location within the model according to execution of a distance function, wherein the location of each entity is updated to provide a finite location within the model.

In some embodiments, the at least one entity can provide a representation of a model derivation having parts of a source model and providing additional updates to distances according to the training of the model.

In addition to the exemplary aspects and embodiments described above, further aspects and embodiments will become apparent by reference to the figures and by study of the following detailed description.

### BRIEF DESCRIPTION OF THE DRAWINGS

Some non-limiting exemplary embodiments or features of the disclosed subject matter are illustrated in the following drawings.

FIG. 1 schematically illustrates a system for generating a multilayered model, according to certain exemplary embodiments;

FIG. 2 shows an exemplary library of databases facilitating the collection of raw data, according to certain exemplary embodiments;

FIG. 3 shows an abstraction of generating and continuously updating the multilayered model, according to certain exemplary embodiment;

FIG. 4 outlines operations for generating a seed model, according to certain exemplary embodiments;

FIG. 5 outlines operations for customizing model according to predetermined requirements, according to certain exemplary embodiments;

FIG. 6 outlines operations for generating an insight in the multilayered model, according to certain exemplary embodiments; and,

FIGS. 7A-7B shows a display of the insight, according to certain exemplary embodiments.

Identical, duplicate, equivalent or similar structures, elements or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views. References to previously presented elements are implied without necessarily further citing the drawing or description in which they appear.

### DETAILED DESCRIPTION

Disclosed herein is a system and method for generating a computational simulations environment for research and design process enhancement as well implementation via having a multi-layer data self evolving knowledge modeling system, according to certain exemplary embodiments.

In the context of the present disclosure, without limiting, raw data is/implies a data type that includes of information that has a defined significance to a field of research and that can be empirically measured via experimental tools and that can be represented via a numerical array.

In the context of some embodiments of the present disclosure, without limiting, an entity is/implies a specific instance of raw data type, that have a unique name, unique and various numerical and textual attributes;

In the context of some embodiments of the present disclosure, without limiting, a layer is/implies a collection of entities having a logical relationship.

In the context of some embodiments of the present disclosure, without limiting, a model or multilayered model is/implies a complete sphere of research having a well-defined collection of layers and functions that is tailored to the needs of a user and facilitates answering questions and obtaining insights, as well as constant machine learning based training of data instances in the system.

In the context of the present disclosure, without limiting, distance is/implies a distance representation of different relationship characteristics between a first entity on a second entity.

In the context of some embodiments of the present disclosure, without limiting, a function is/implies an analysis of the entities and/or their relationship that describes a mathematical equation represented according to the distance between the entities.

In the context of the present disclosure, without limiting, an insight is/implies a technologically significant output having a visual and textual representation of the raw data, entity function and/or their combinations of which are derived from the model.

The terms cited above also denote inflections and conjugates thereof.

A multilayered interactive research model or a multilayered interactive research map provides a data-embedding translation and fusion system with an interface generated through interactive machine learning supported by a data-fusion engine. The system is configured to provide continuous and constant data transfer, data fusion and data evaluation between different types of data published by the science and technology community, for example the biomedical community, and between users in the research, design and implementation communities, such as the medical community. The constant improvement and analysis of the collected data enhances the ability of researchers and other analysts, to derive insights relating to the collected data that can have an important effect on the advancement of the technological or scientific field.

The fusion of all data types in a particular data analysis field is a world-wide recognized challenge. The main paradigm is that each data type of any specific technologically related relevant data-environment is available in a specific and/or unique definition or description within the specific field of research or technology, thereby having a particular research world of data. The system and method disclosed herein for generating a model that provides continuous and automatic computational data analysis of the data in the model. The system and method connect and determine the relationships between the various data types in the model by assigning ‘locations’ to the data within the model and placing the data in the same model to facilitate research quality and quantity in any particular field.

The model, also referred to as a map, is a data structure that represents a specific field of study environment. In some embodiments, a medical or biological field of study environment can represent individual cancer cells undergoing experiments that cause them to undergo oxygen deficiency processes, a physiological environment of gastrointestinal tissues in diabetics, who receive swallowing drugs, a stem cell research environment that receives molecules and then implants an environment of tissues in the gut that make up the cells receive small molecules that alter neuronal processes and/or the like. The field of study environment represents a backlog of relatively close experiments, medical treatments, related articles, and/or the like. In some embodiments, the field of study environment may include one cell, a number of cells, a number of tissues or any particular subsystem of the human body undergoing a particular experiment. Thus, all field of study environments include the same instances for the same data system, with different values and/or measurements in which each environment can include the same data type. For example, hundreds environments can represent hundreds of thousands of users.

The system and method disclosed herein can locate all of the elements that represent a particular user or field of study environment within unifying data interactions that are modeled in a customizable manner that facilitates the interaction of the user with the model and facilitates creating new elements in the model. Following such location of any data type, to the same unifying system, the data model provides a uniform platform for the presentation, in a unifying manner, of the information thereby reducing the problem in customization of the information, thereby reducing analysis time and increases the reliability of the information. In some embodiments, the models are not merely measurements of separated data entities that are ‘located’ and unified. Each model describes a specific environment that includes a collection of effects and interactions that exist among entities presented in the model. Thus, model creates a fusion of the ability to describe and characterize the interplay of the entities and can be presented to a user in a user-friendly interactive manner. In some exemplary embodiments, the system and method are implemented to create a full biological model showing a drug-disease biological environment that can help a user answer crucial research questions regarding the interaction and relationship between drugs, diseases, drug effects and/or the like, in a way that relies on the mode of action of the drug-disease mechanism. In certain exemplary embodiments of data spaces, the drugs, the diseases, the proteins, the genes and the effects between them can be described through implementation of the unified system of data disclosed herein. For example, reference to both drugs, diseases, genes and proteins is described through the interactions between proteins only. For example, the model can describe thousands of rich-data drug-disease biological environments using only one and the same data-type.

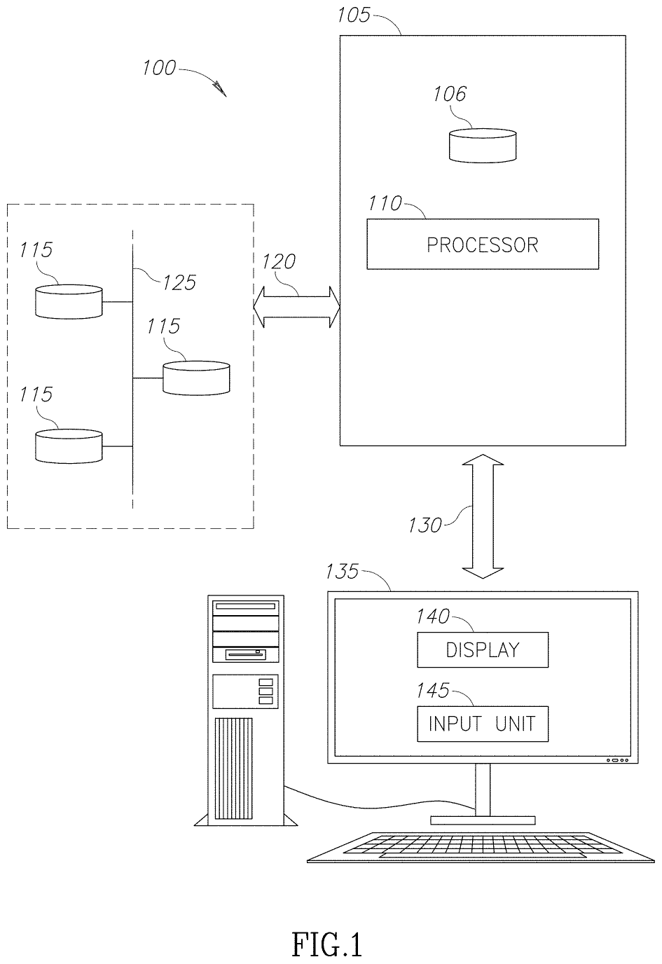

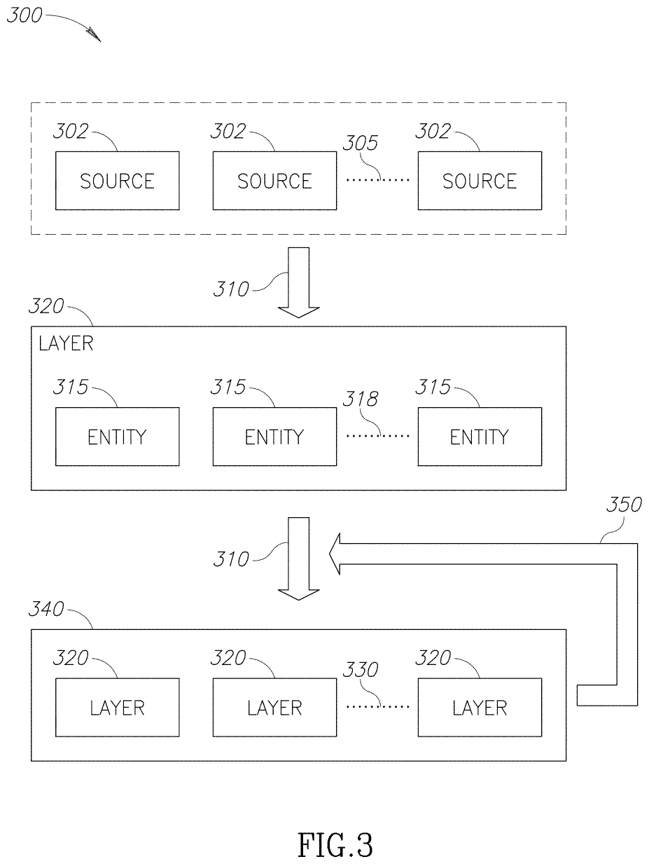

Reference is now made to FIG. 1, schematically illustrating a system 100 configured for generating a multilayered model 340 (FIG. 3), according to certain exemplary embodiments. System 100 includes a mapping server 105, executing a computer program product, the computer program product includes a non-transitory computer-readable storage medium having program code embodied therewith, the program code executable by at least one hardware processor 110.

System 100 includes one or more databases 115, illustrated as three instances of a database 115, representing any number of databases 115, as indicated by dashed line 125. Mapping server 105 is connected or linked to databases 115 by any communication facility or facilities included in system 100 as schematically illustrated by arrow 120, which facilitate the data flow from one or more databases 115 to mapping server 105.

One or more databases 115 store data with which mapping server 105 generates the multilayered model 340. The data, also referred to as raw data, includes information that has significance to a predetermined field of study. By way of example, the raw data is available in articles, patent literature, medical records, experimental data, schematics, technical journals, and/or the like. In some exemplary embodiment, the raw data relates to raw biological data including information relating to genes, proteins, metabolites, medical test results, and/or the like detailed and described in articles, patient medical charts, experiments and/or the like.

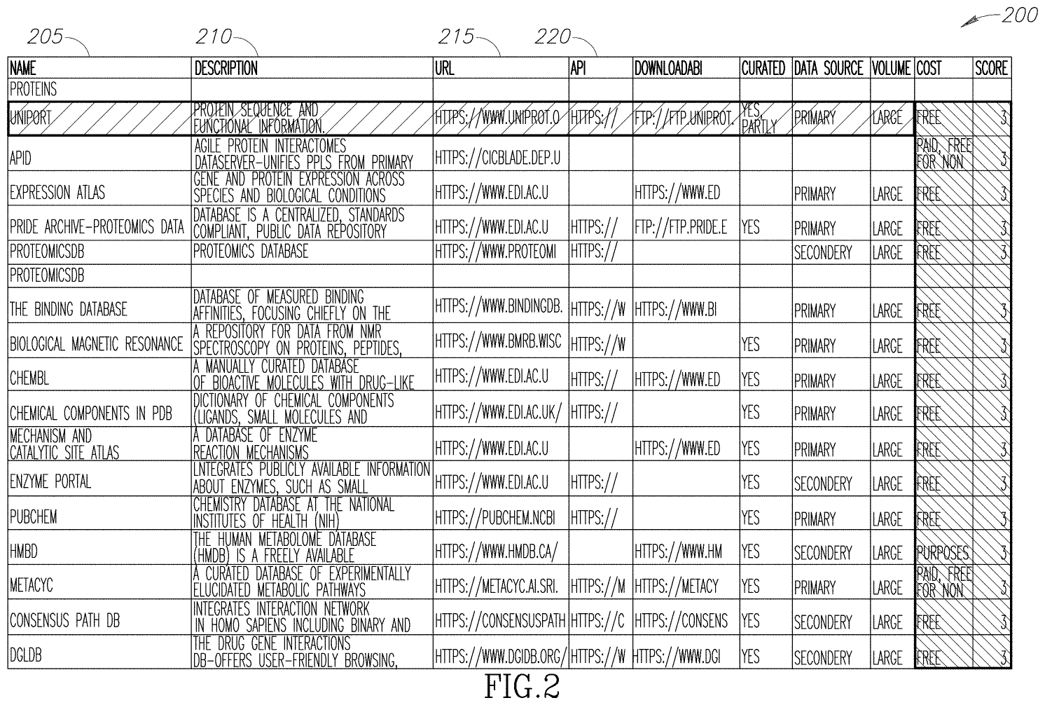

Mapping server 105 includes a memory unit 106 for storing a database library 200 (FIG. 2), a database list, and or the like. Memory unit 106 stores a multilayered model 340 or map of the field of study environment to which multilayered model 340 is related.

Processor 110 is configured to analyze and generate the model 340 through execution of predetermined modules and operations. In some embodiments, processor 110 executes database crawling to continuously and automatically obtain the raw data stored in databases 115 storing in one or more sources 302 (FIG. 3). Links or access routes to databases 115 are listed in database library 200 (FIG. 2). Database crawling enables processor 110 to obtain the raw data and then implement a parsing module to the raw data, thereby breaking down the raw data into predetermined structures and/or to tokenize the raw data to provide parsed data.

In some embodiments, processor 110 executes an entity extraction module to the parsed data to extract one or more entities 315 (FIG. 3) from the parsed data according to one or more terms that are seeded in mapping server 105 or that are provided to mapping server 105 from an external source. In some embodiments, after extraction of entities 315, processor 110 can verify the entities 315 to ensure that they are relevant to the field of study environment for which the model is generated.

Processor 110 executes a function module that implements one or more functions to the raw data and the entities 315 to establish different relationship parameters between the entities 315. In certain embodiments, processor 110 can implement a distance function, an insight function, a compatibility function, and/or the like. The function module includes a train function for providing an analysis of the relationship between entities according to real or virtual experiment results for generating model 340.

Processor 110 executes a layer generating module to generate one or more layers 320 (FIG. 3) that define the relationships between the entities 315 and generates a space representation of the relationship. The space representation can be implemented by providing a location of an entity 315 (FIG. 3) within the space representation and a distance between a location of a first entity to a second entity. Each layer 320 can represent one or more relationships between the entities 315 and can provide the raw data relating to the relationships. By way of example, in a biological field of study, a layer can include a protein to protein interaction layer, an articles layer, a patents layer and/or the like.

Processor 105 is configured to execute a model generating module, which generates the multilayered model 340 including the raw data, entities 315 and layers 320 associated with the field of study. The model 340 provides a graphical representation of the model 340, thereby facilitating a research to view and interact with the model 340.

In certain embodiments, the function module includes an insight module for evaluating the data in the multilayered model 340 to determine a best match between entity 315 and a desired repurposing according to the raw data, there by generating a new application of the entity. By way of example, applying an insight function to a drug and finding a repurposing of the drug through analysis of the raw data and then providing this new application to be visually displayable in multilayered model 340.

It is appreciated by one skilled in the art, that as databases are added to database library 200, database crawling is executed on those additional databases to access their data thereby enabling mapping server 105 to update model 340.

In certain embodiments, mapping server 105 can cluster specific databases 115 listed in database library 200 according to common information and/or a designated fields of study. The clusters can be ranked according to preference to a researcher by assigning a priority value to each cluster according to the relevance of entity 315 and/or layer 320 of model 340.

System 100 includes a user computer 135, which is connected or linked to mapping server 105 by any facility or facilities included in system 100, as schematically illustrated by arrow 130, which facilitates the data flow between user computer 135 and mapping server 105. In some embodiments, user computer 135 is generally operated by professionals thereby enhancing the improvement of the model 340 by providing mapping server 105 with new data and/or inputs. For example, in the field of biology, the professionals can be doctors, pharmacists, drug researchers, university researchers, and/or the like, that provide new experimental data, patient charts, drug recipes, and/or the like.

User computer 135 includes a display 140 configured to enable user computer 135 to display model 340 in the form of a graphical map. User computer 135 includes an input 145 for obtaining an input from a user of the user computer 135 and enables the user to interact with model 340. In certain embodiments, user computer 135 includes a graphic user interface 150, which includes display 140 and input 145. The user of the user computer 135 can access and interact with model 340 through the connection between mapping server 105 and user computer 135.

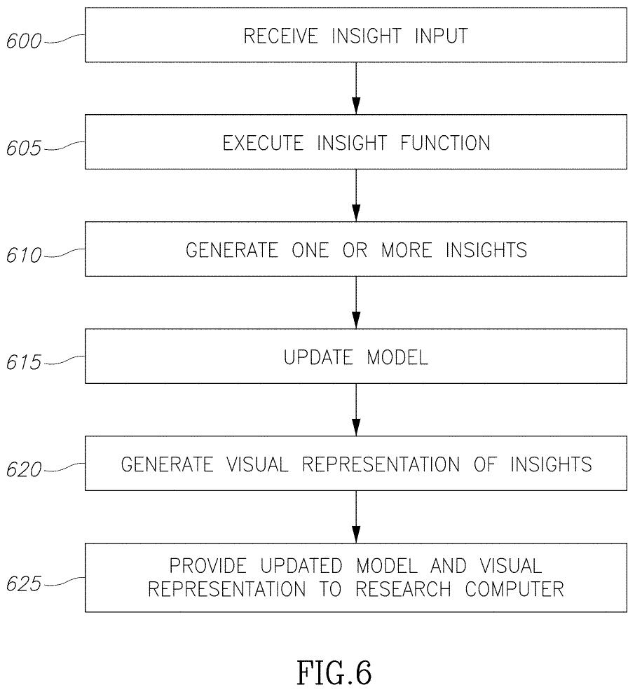

In certain embodiments, mapping server 105 is configured to provide a data analysis that is derived through compared prediction performance upon various types of potential hypothesized and theoretical interactions network and that are not derived from any real world-experimental quality related estimation system. In certain embodiments, mapping server 105, as described in conjunction with FIGS. 3-6, provides a comparison, evaluation, reliability determination and relative importance of any entity 302 relative to other entities in the model 340. In certain embodiments, mapping server 105, as described in conjunction with FIG. 6, provides a comparison, evaluation and effectiveness of data insight displaying methodology of data relative to related mathematically and empirically elements. In some embodiments, mapping server 105 is configured to combine measurements, insights types and attributes through combination of entities 315 of a field of study, thereby finding new potential terminologies for the combinations according to data acquired from patent literature, patient data, articles, and/or the like. In certain embodiments, mapping server 105 is configured to generate mathematical and/or computational definitions of an optimal location of entities 315 within model 340 within a predetermined scope of a field of study. For example, entities within a scope of researchers, experiments and/or the like.

FIG. 2 shows a database library 200 having a list of database addresses to facilitate obtaining the raw data from databases 115 (FIG. 1), according to certain exemplary embodiments. Database library 200 can be in the format of a chart, or table listing a database name 205, a database description 210, a database URL link 215, a database API link 220 and/or the like. Processor 110 (FIG. 1) executes the database crawling according to the database library 200 thereby extracting the raw data from the databases 115.

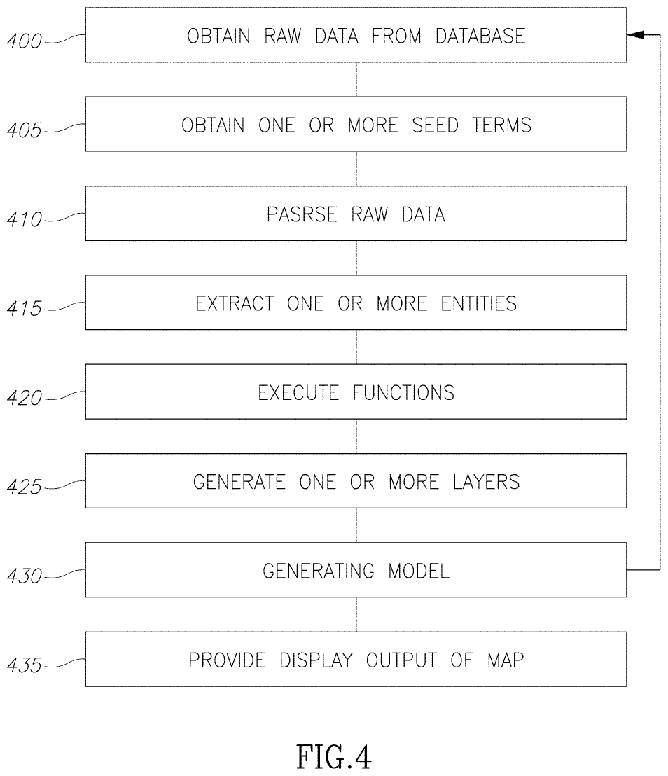

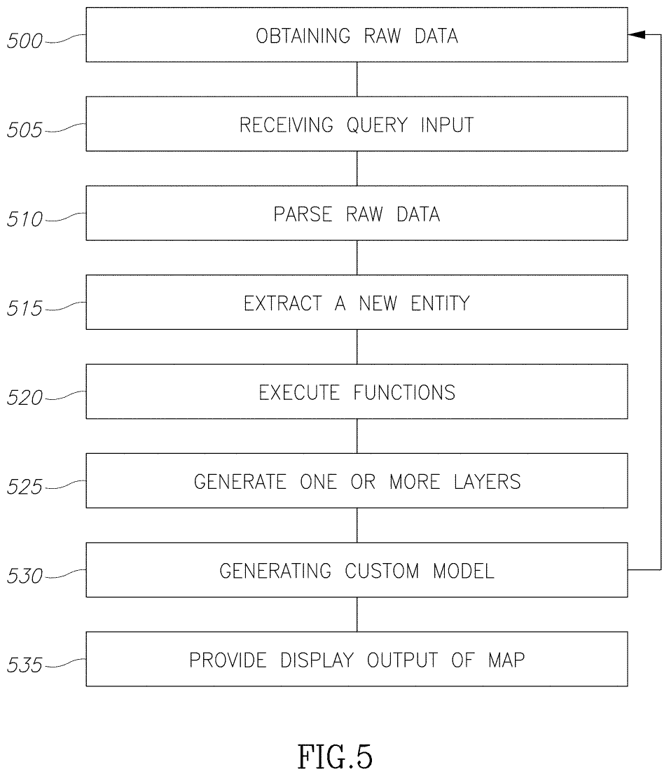

FIG. 3 shows a visual abstraction of generating a model 340, according to certain exemplary embodiment. Mapping server 105 (FIG. 1) obtains data, for example, raw data, from one or more sources 302 illustrated by three instances of sources 302, representing any number of sources 302, as indicated by dashed line 305. One or more sources 302 can be stored in one or more databases 115 (FIG. 1) and can include different types of sources, such as real or simulated experiments, articles, technical charts, patent literature, and/or the like. Mapping server 105 generates a layer 320, as represented by arrow 310. Layer 320 includes one or more entities 315, illustrated by three instances of entities 315 representing any number of entities 315, as indicated by dashed line 318. One or more entities 315 are extracted by mapping server 105 from the data obtained from one or more sources 302. Mapping server 105 generates a multilayered model 340 including one or more layers 320, illustrated by three instances of layers 320, representing any number of layers 320, as indicated by dashed line 325. As disrobed in conjunction with FIGS. 4-5, model 340 is continuously trained, represented by arrow 350, to update model 340 with additional layers 320 and entities 315. Training of model 340 is performed through execution of the training function, which is a continuous and automatic analysis of the mathematical relationships between entities 315, layers 320 and/or the like. The train function then modifies and/or updates the distance between the entities 315 and can provide additional information relating to the relationship and characteristics of the interactions between the entities 315 and layers 320 in model 340. In some embodiments, the continuous training is form mapping server 105 obtaining new raw data, or new terms that are relevant to the field of study. In some embodiments, mapping server 105 learns to extract new entities according to existing entities and layers.

FIG. 4 outlines operations for generating model 340 (FIG. 3), according to certain exemplary embodiments. In operation 400, mapping server 105 (FIG. 1) obtains the raw data from databases 115 (FIG. 1) through database crawling the databases 115 listed in database library 200 (FIG. 2). Obtaining the raw data can be from different sources 302 (FIG. 3) that are stored in databases 115, as described in conjunction with FIG. 3.

In operation 405, mapping server 105 obtains one or more seed terms for executing a parsing of the raw data to generate parsed data. The one or more seed terms can be provided as an initial list of terms associated with a field of study. By way of example, in the field of biology, terms can include protein names, drug name, diseases and/or the like.

In operation 410, mapping server 105 parses the raw data into smaller portions to facilitate extraction of entities 302 (FIG. 3). In certain embodiments, parsing of the raw data can include tokenizing the raw data.

In operation 415, mapping server 105 extracts one or more entities 302 from the raw data according to the one or more seed terms. In some embodiments, entities 302 are extracted from the parse data according to the one or more seed terms.

In certain embodiments, mapping server 105 validates the entities 302 while extracting the entities 302 thereby verifying that the extracted entities 302 are relevant to the field of study for which model 340 is constructed. For example, an extracted entity 302 can include the term electricity, but because it is not relevant to the field of study of biology, it is rejected and not included with the validated entities.

In operation 420, mapping server 105 executes one or more functions to determine an effect or interaction between the one or more entities 302. The one or more functions define a relationship between the entities 302, for example a distance relationship, an interaction relationship, an effect relationship and/or the like between the one or more entities 302.

In operation 425, mapping server 105 generates one or more layers 320.

In operation 430, mapping server 105 generates model 340. Mapping server 105 executes a training function to generate the model 340 by updating the entity relationships according to real or virtual experiments and/or other data. The model 340 is an initial model providing an initial relationship between the entities 302 generated by user computer, where each relationship and entity 302 can be part of layer 320 in the seed model. After model 340 is generated, mapping server 105 continuously updates and modifies model 340 according to retraining as discussed in conjunction with FIG. 3.

In operation 435, mapping server 105 provides model 340 to user computer 135. Once received by user computer 135, the user of user computer can view and interact with model 340.

FIG. 5 outlines operations for generating a custom model 340, according to certain exemplary embodiments.

In operation 500, mapping server 105 (FIG. 1) obtains the raw data. The raw data is obtained through data crawling of the one or more databases 115 according to database library 200 (FIG. 2).

In operation 505, mapping server 105 obtains query input. The query input can be received from user computer 135 at which the user has provided one or more terms that are to be used to extract entities from the raw data. In certain embodiments, the query input can include information about the user, for example, a field of study of the user, past experiments performed by the user, articles written by the user, and/or the like.

In operation 510, mapping server 105 parses the raw data to obtain the parsed data as disclosed in conjunction with FIG. 1.

In operation 515, mapping server 105 extracts new entities from the parsed data.

In operation 520, mapping server 105 executes one or more functions to determine a relationship between the new entity and other entities 302 (FIG. 3) in the model 340.

In operation 525, mapping server 105 generates the one or more layers 320 (FIG. 3). Each layer 320 includes a collection of entities 302 having a predetermined logical relationship between them. The logical relationship can be determined according to implementation of a distance function, a location function, and/or the like.

In operation 530, mapping server 105 generates a custom model to provide a model that includes the entities and relationships generated in accordance with the query inputs. The train function is executed to generate custom model and facilitates analyzing the relationship between predetermined entities to determine new information about the relationship between the predetermined entities through analysis of real or virtual experiments results as well as newly discovered information. After implementation of the train function, mapping server 105 can execute the distance function in operation 520 to realign and determine the distance between the predetermined entities.

In operation 535, mapping server 105 provides the custom model to the user computer 135 to enable the user to interact and view the custom model.

In certain exemplary embodiments, model 340 (FIG. 3) is updated according to the new entities generated by the query input, thereby model 340 is customized for use by a particular researcher according to the query input that is provided.

FIG. 6 outlines operations for generating an insight output, according to certain exemplary embodiments. In operation 600, mapping server 105 receives an insight input, which includes a query to extract an insight regarding a relationship between entities 302 (FIG. 3) in model 340 (FIG. 3).

In operation 605, mapping server 105 executes an insight function on model 340. The insight function derives insights on the raw data that is most important to research decisions, thereby enhancing the quality of the research. The insight function further analyzes the data to determine an optimal repurposing of one or more entities 302 (FIG. 3) in the model 340. By way of example, the insight function analyzes the data and the entities 302 to determine whether a particular drug can be repurposed for treatment of a disease for which the drug is currently not intended.

In operation 610, mapping server 105 generates one or more insights according to the results of the insight function.

In operation 615, mapping server 105 updates model 340 to include the one or more insights.

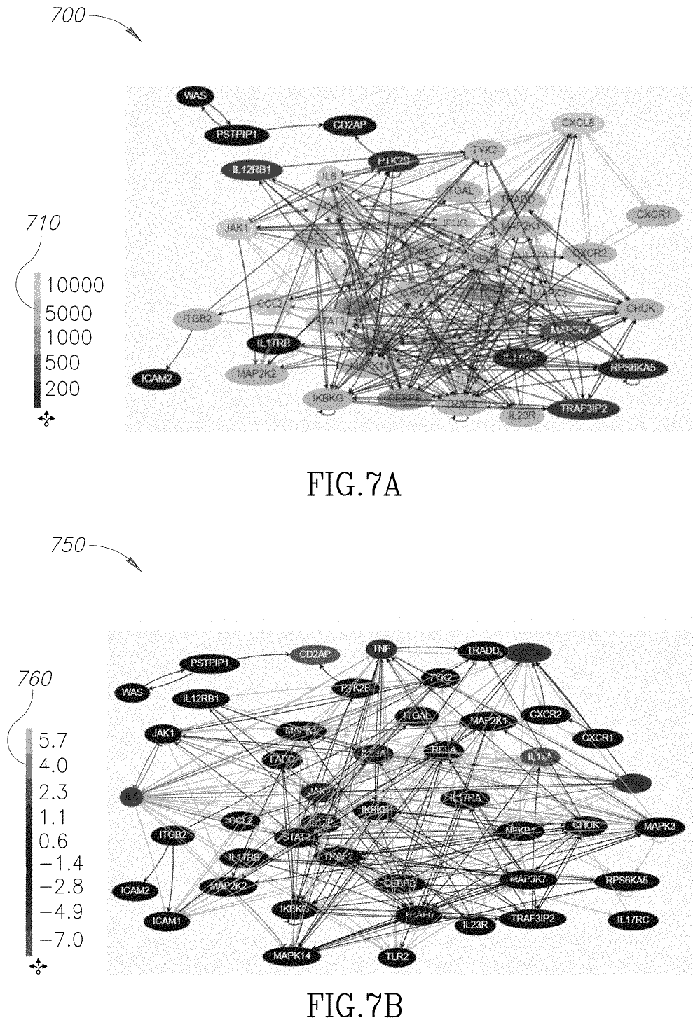

In operation 620, mapping server 105 generates a visual representation of the one or more insights by computer 135. The visual representation is a generated according a mathematical relationship of the insights and entities 302. The insight between entities 302 can be represented as lines and distance between one or more entities 302, as described in conjunction with FIGS. 7A-7B.

In operation 625, mapping server 105 provides the updated model including the one or more insights to the user computer 135 (FIG. 1), thereby enabling the user to view and interact with the one or more insight outputs.

In some embodiments, mapping server 105 is configured to obtain data relating to a product from which it can determine a mechanism of the product, thereby utilizing the mechanism to produce a recommended usage of the product according to the insight. The product provide can provide a use in a predetermined field of study. For example, the product can be a software, a drug, a widget and/or the like that in its respective field of study provides some beneficial use. Mapping server 105, through the insight, determines a new productive use of the product that might not have been previously known or considered

FIGS. 7A-7B shows a display of the insight, according to certain exemplary embodiments. FIG. 7A shows a display 700 of article mapping according to insight relationships between different entities presented in the model 340 (FIG. 3). The lines between entities represents the distance between the entities, and a color mapping can show a relevance or effect of each entity on one another according to what is disclosed in the article. A color scale 710 can be provided to assist in determining the relationship importance of each entity. The map is provided to user computer 135 (FIG. 1) to provide a user with possible uses or effects of certain entities on one another. For example, showing the possible effect of an experimental drug on a predetermined disease. As shown, the entities are displayed as nodes having pathways connecting the nodes. A weight is provided to the pathways according to the relationship of the entities. For example, the effect of predetermined drugs on a disease, the most efficient drugs will have pathways in a color that emphasizes the relationship. The pathways are updates according to the calculations and updating resulting from the retraining of the model and modifying the distance and/or other relationship characteristics between the entities. In some embodiments, nodes can expand to show a chart or graph of the characteristics and effects the entity exhibits on other entities in the model. In some embodiments, the nodes represent the articles or data source in which the entity is disclosed and the pathways show the relevance of the article to other articles discussing the entity.

FIG. 7B shows a display 750 of gene mapping according to insight relationships between different entities presented in the map. A color scale 760 can be provided to assist in determining the relationship importance of each entity.

By way of non-limiting example, mapping server 105 can be implemented to find a relationship between an existing pharmaceutical drug and a disease. The drug and disease name are provided to mapping server 105, for which mapping server 105 obtains synonyms and related materials through data crawling of databases 115 and obtaining the related raw data. Mapping server 105 calculates the significant proteins in the drug and additional data is obtained from the database after which mapping server 105 generates the relevant entities, layers and model. An inquiry input is provided from which seed data relating to the drug and disease are used generate related relationships and issues between the drug and disease. For example, defining a limited number of proteins, biological phenomena, diseases, and/or the like are issues and relationships that are analyzed by mapping server 105.

Mapping server 105 implements the distance function and thereby extracts, for example, the most relevant proteins found in the disease and in similar diseases. After determining the distance, the train and insight functions are implements to determine an influence of the drug on the disease, any side effects, such as inflammatory mechanism, and relevant supplementary information, such as patents, articles, or the like. Mapping server 105 updates the model with the new information and generates the necessary new layers accordingly. All of the information is provided to user computer 135 (FIG. 1) to enable a user to review the material and interact with the model.

By way of another example, mapping server 105 is provided with a patient data, for example, blood tests, genetic background, disease background, and/or the like. Mapping server 105 generates entities from the provided data, such as convert blood tests into biomechanical effects, convert genetic background to active Single-Nucleotide Polymorphisms (“SNPs”), and/or the like. Mapping server 105 determines a distance between the current patient and an average patient with the current condition. Mapping server 105 generates the layers and trains the data on a set of patients and successful treatments which effects are known thereby by updating the distance and the layers. The mapping server 105 receives an inquiry input, for example a question of whether a specific treatment is appropriate for the patient after which mapping server 105 and implementing the insight function to provide an inference score, an insight and relevant supplementary information, which is generated, the model is updated and the model is provided to user computer 135 for viewing and interaction.

In the context of some embodiments of the present disclosure, by way of example and without limiting, terms such as ‘operating’ or ‘executing’ imply additional capabilities, such as ‘operable’ or ‘executable’, respectively. Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry. The term computerized apparatus or a computerized system or a similar term denotes an apparatus comprising one or more processors operable or operating according to one or more programs. As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome. As used herein, the term ‘server’ denotes a computerized apparatus providing data and/or operational service or services to one or more other apparatuses. The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium. In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof. As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value. Unless otherwise specified, the terms ‘about’ and/or ‘close’ with respect to a dimension or extent, such as length, implies within an inclusive range of −10% to +10% of the respective dimension or extent. Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively. The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded. Terms in the claims that follow should be interpreted, without limiting, as characterized or described in the specification.

1. A method comprising using at least one hardware processor for:
obtaining raw data relating to at least one predetermined field of study;
obtaining at least one term;
generating at least a first entity and a second entity from said raw data according to said at least one term;
implementing a predetermined function to determine a relationship between said first and second entities;
generating a layer aligning said first and second entities according to the relationship between said first and second entities; and,
constructing a model of data-interactions according to said at least one interactive map layer.

2. A method according to claim 1, wherein implementing said predetermined function comprises implementing a training function configured to determine a relationship between at least two entities via computational simulations or other mathematical translational method in view of experimental data.

3-5. (canceled)

6. A method according to claim 1, wherein said model comprises a complete research collection having a well-defined collection of entities, layers and functions customized to a requirement of a user.

7. A method according to claim 1, wherein said predetermined function is a distance-function for determining mathematical representations of cause- and effect measurements, in a unifying manner between said first and second entity.

8-13. (canceled)

14. A method according to claim 1, further comprising:
obtaining data relating to a product;
determining a mechanism of the product; and,
utilizing a research mechanism to produce a recommended usage of the product according to said at least one insight.

15. A method according to claim 1, further comprises executing a train function to automatically and continuously update the model with according to new data and inputs.

16-19. (canceled)

20. A computer program product for generating an interactive data-transfer and analysis computational model, the computer program product comprising a non-transitory computer-readable storage medium having program code embodied therewith, the program code executable by at least one hardware processor to:
obtain raw data relating to at least one predetermined field of study;
obtain at least one term;
generate at least a first entity and a second entity from said raw data according to said at least one term;
implement a predetermined function to determine a relationship between said first and second entities;
generate a layer aligning said first and second entities according to the relationship between said first and second entities; and,
construct a model of data-interactions according to said at least one interactive map layer.

21. A computer program product according to claim 20, wherein implementing said predetermined function comprises implementing a training function configured to determine a relationship between at least two entities via computational simulations or other mathematical translational method in view of experimental data.

22-24. (canceled)

25. A computer program product according to claim 20, wherein said model comprises a complete research collection having a well-defined collection of entities, layers and functions customized to a requirement of a user.

26. A computer program product according to claim 20, wherein said predetermined function is a distance-function for determining mathematical representations of cause- and effect measurements, in a unifying manner between said first and second entity.

27-32. (canceled)

33. A computer program product according to claim 20, further comprising:
obtaining data relating to a product;
determining a mechanism of the product; and,
utilizing a research mechanism to produce a use recommendation of the product according to said at least one insight.

34. A computer program product according to claim 20, further comprises executing a train function to automatically and continuously update the model with according to new data and inputs.

35-38. (canceled)

39. A system comprising:
at least one database for storing raw data;
a mapping server configured to:
obtain raw data relating to at least one predetermined field of study;
obtain at least one term;
generate at least a first entity and a second entity from said raw data according to said at least one term;
implement a predetermined function to determine a relationship between said first and second entities;
generate a layer aligning said first and second entities according to the relationship between said first and second entities; and,
construct a model of data-interactions according to said at least one interactive map layer;
and,
a computer having a user interface for displaying said model and to enable a user to interact with said model.

40. A system according to claim 39, wherein implementing said predetermined function comprises implementing a training function configured to determine a relationship between at least two entities via computational simulations or other mathematical translational method in view of experimental data.

41-43. (canceled)

44. A system according to claim 39, wherein said model comprises a complete research collection having a well defined collection of entities, layers and functions customized to a requirement of a user.

45. A system according to claim 39, wherein said predetermined function is a distance-function for determining mathematical representations of cause- and effect measurements, in a unifying manner between said first and second entity.

46-51. (canceled)

52. A system according to claim 39, wherein said mapping server is further configured to:
obtain data relating to a product;
determine a mechanism of the product; and,
utilize a research mechanism to produce a use recommendation of the product according to said at least one insight.

53. A system according to claim 39, wherein the mapping server is further configured to execute a train function to automatically and continuously update the model with according to new data and inputs.

54-57. (canceled)
